(19) United States
(12) Patent Application Publication
(10) Pub. No.: US 2025/0276134 A1
(43) Pub. Date: Sep. 4, 2025

(54) OPTIMIZING CONTACT FORCE FOR AEROSOL DELIVERY

(71) Applicant: PNEUMA RESPIRATORY, INC., Boone, NC (US)

(72) Inventors: Charles Eric HUNTER, Boone, NC (US); Jeffrey MILLER, Boone, NC (US); Chao-Ping LEE, Kaohsiung (TW); Chengjie LI, Shenzhen (CN); Jianqiang LI, Shenzhen (CN)

(73) Assignee: PNEUMA RESPIRATORY, INC., Boone, NC (US)

(21) Appl. No.: 19/070,274

(22) Filed: Mar. 4, 2025

Related U.S. Application Data
(60) Provisional application No. 63/701,563, filed on Sep. 30, 2024.
(60) Provisional application No. 63/561,072, filed on Mar. 4, 2024.

(51) Int. Cl. A61M 11/00; A24F 40/05; A24F 40/10; A24F 40/40; B05B 17/06

(52) U.S. Cl. CPC A61M 11/005; A24F 40/05; A24F 40/10; A24F 40/40; B05B 17/0607; A61M 2205/0294

(57) ABSTRACT

An aerosol delivery device includes a piezoelectric transducer that is indirectly coupled to an ejector plate, such as by a membrane between the transducer and ejector plate. A spring force is applied between the ejector plate and transducer in different configurations by a suspension gasket, a compression spring, or flat spring. The spring force optimizes the production and ejection of droplets from fluid supplied to the ejector plate when the transducer vibrates to produce aerosol from the device.

### CROSS-REFERENCE TO RELATED APPLICATIONS

This application claims the benefit of U.S. Provisional Pat. App. No. 63/701,563 filed Sep. 30, 2024, and U.S. Provisional Pat. App. No. 63/561,072 filed Mar. 4, 2024, which are incorporated herein by reference in their entirety.

### FIELD OF THE INVENTION

This disclosure relates to droplet delivery devices with ejector mechanisms and more specifically to droplet delivery devices for the delivery of fluids that are inhaled into mouth, throat, nose, and/or lungs.

### BACKGROUND OF THE INVENTION

The use of droplet generating devices for the delivery of substances to the respiratory system is an area of large interest. A major challenge is providing a device that delivers an accurate, consistent, and verifiable amount of substance, with a droplet size that is suitable for successful delivery of substance to the targeted area of the respiratory system.

Currently most inhaler type systems, such as metered dose inhalers (MDI), pressurized metered dose inhalers (p-MDI), or pneumatic and ultrasonic-driven devices, generally produce droplets with high velocities and a wide range of droplet sizes including large droplets that have high momentum and kinetic energy. Droplet plumes with large size distributions and high momentum do not often reach targeted locations of the respiratory pathway.

Droplet plumes generated from current droplet delivery systems, as a result of their high ejection velocities and the rapid expansion of the substance carrying propellant, may also lead to localized cooling and subsequent condensation, deposition and crystallization of substance onto device surfaces. Blockage of device surfaces by deposited substance residue is also problematic.

Further, conventional droplet delivery devices for delivery of nicotine, including vape pens and the like, typically require fluids that are inhaled to be heated to temperatures that negatively affect the liquid being aerosolized. Specifically, such levels of heating can produce undesirable and toxic byproducts as has been documented in the news and literature.

Accordingly, there is a need for an improved droplet delivery device that delivers droplets with improved consistency and reproducibility.

### SUMMARY OF THE INVENTION

In one example, an aerosol delivery device includes a piezoelectric transducer indirectly coupled to an ejector plate and a suspension gasket coupled to the ejector plate that is configured to apply spring force between the ejector plate and the transducer.

In another example, an aerosol delivery device includes a piezoelectric transducer indirectly coupled to an ejector plate and a compression spring coupled to the piezoelectric transducer and configured to provide a spring force between the ejector plate and the transducer. In some examples the ejector plate is held by a suspension gasket configured to provide additional spring force between the ejector plate and the transducer. In further examples the ejector plate is rigidly held in place and wherein only the compression spring is providing spring force.

In another example, an aerosol delivery device includes a piezoelectric transducer indirectly coupled to an ejector plate and a flat spring coupled to the ejector plate that is configured to apply contact force between the ejector plate and the transducer.

In another example, an aerosol delivery device includes a piezoelectric transducer indirectly coupled to an ejector plate and one or more constant force springs coupled to the piezoelectric transducer and configured to provide a contact force between the ejector plate and the transducer. In some examples the ejector plate is held by a suspension gasket configured to provide additional spring force between the ejector plate and the transducer. In further examples the ejector plate is rigidly held in place wherein only the constant force spring or constant force springs provide spring force.

In another example, an aerosol delivery device includes a piezoelectric transducer indirectly coupled to an ejector plate via a membrane between the transducer and the plate, wherein the membrane includes folds configured to control contact force on the membrane.

In another example, an aerosol delivery device includes a piezoelectric transducer indirectly coupled to an ejector plate via a membrane between the transducer and the plate, wherein spring force is being applied between the ejector plate and the transducer; a vibrating member between the membrane and the plate, and a desiccant placed near the vibrating member that is configured to absorb unwanted moisture. In some examples, the desiccant is housed in a location sealed from the environment.

### BRIEF DESCRIPTION OF THE DRAWINGS

FIG. 1 is a graph and table showing data from contact force testing. Seven ejector sets were tested at varying contact forces. The first row of values in the table is the x-axis values. The rest of the data in the table is the measured ejected mass at that contact force.

FIG. 2 is a cross-sectional view of one example of a droplet delivery device including a push mode design including components facilitating contact force and ejection.

FIG. 3 is a cross-sectional view of one example of a droplet delivery device including an ejector assembly that moves onto the vibrating member assembly in a push mode design. The vibrating member tip contacts the membrane ejector plate interface.

FIGS. 4A and 4B are cross-sectional views of one example of a droplet delivery device including three-degree angle that is in a push mode design. The higher stop and lower stop force the ejector plate to be at a three-degree angle. The suspension gasket allows for the three-degree angle.

FIG. 5 is a cross-sectional view of one example of a droplet delivery device including a push mode device where the vibrating member has too much force against the ejector assembly. It causes the ejector plate to become flat against the vibrating member tip. This impedes the vibrations on the ejector plate and limits the amount of ejection.

FIG. 6 is a cross-sectional view of one example of a droplet delivery device including a push mode device where the vibrating member has too little force against the ejector assembly. At the boundary case, there will be no contact at all between the vibrating member tip and the ejector plate.

FIG. 7 is a cross-sectional view of one example of a droplet delivery device including measurement of tensile force against the membrane as a quality check in a push mode device. A vibrating member tip is forced onto the membrane at a known location and the force is measured.

FIG. 8 is a cross-sectional view of one example of a droplet delivery device including measurement of tensile force against the membrane and ejector plate as a quality check in a push mode device. A vibrating member tip is forced onto the ejector assembly at a known location and the force is measured.

FIG. 9 is a perspective view of one example of a droplet delivery device membrane design. There is additional material added at the angle point of the design, which is shown. The extra material is similar to one step in an accordion.

FIG. 10 is a perspective view of one example of a droplet delivery device membrane that does not have extra material to minimize contact force.

FIG. 11 is a cross-sectional view of one example of a droplet delivery device illustrating a location on the membrane that gets too tight when there is too much contact force between the vibrating member and the membrane. This point will experience excess vibrations and increased friction. The increase in friction will cause heat and will damage the membrane. Limiting the contact force is necessary for the longevity of the membrane.

FIG. 12 is a cross-sectional view of examples illustrating additional droplet delivery device membrane designs to help mitigate additional forces on the membrane. The accordion steps will relieve some of the stress from any force applied to the membrane from the vibrating member.

FIG. 13A is a perspective view and 13B is a cross-sectional view of one example of a droplet delivery device including a spring beneath the vibrating member. The spring forces the vibrating member up to the ejector assembly. The spring can include a low spring constant such that the force applied to the ejector assembly stays within an acceptable range.

FIG. 14 is a perspective view of one example of a constant force spring (Lee Spring of Brooklyn, New York) that may be used in examples of droplet delivery devices.

FIG. 15 is a cross-sectional view of one example of a droplet delivery device including a constant force spring incorporated into a push mode design. An assembly attached to the housing of the vibrating member is attached to the end of the constant force spring. The center of the constant force spring is attached to the handpiece. The constant force spring wants to coil itself back up. This forces the vibrating member into the ejector assembly. The force at which the spring provides does not change based upon the distance it is displaced.

FIG. 16 is a cross-sectional view of one example of a droplet delivery device suspension gasket and ejector plate.

FIG. 17 are perspective views of examples of flat springs (see “3dmodelspace.com”)

FIG. 18 is a cross-sectional view of one example of a droplet delivery device including a desiccant ring that can attached to the ejector assembly on the outside of the membrane.

FIG. 19 are cross-sectional views of one example of a droplet delivery device including a fixed ejector plate. The ejector plate is fixed in place and does not move when the vibrating member tip comes in contact with it. There are two designs, one where a bottom ring is fixed to hold the ejector plate in place and one where a top ring is fixed to hold the ejector plate in place.

FIG. 20 are cross-sectional views of multiple examples of droplet delivery device suspension gaskets. One advantageous example is design “C”.

FIG. 21 are graphs showing the contact force (y-axis in N) as the position of the vibrating member tip changes (x-axis in mm). Every test was done with design “C” shown in FIG. 20. Each plot is a different durometer of silicone. The desired position of the vibrating member is 0.00 mm. The lower the durometer, the more stabile the contact force.

### DETAILED DESCRIPTION

This disclosure incorporates herein by reference in their entireties the disclosures of U.S. Pat. No. 11,793,945, entitled “Droplet Delivery Device with Push Ejection,” and U.S. Provisional Pat. App. Nos. 63/561,072 and 63/701,563 entitled “Optimizing Contact Force for Aerosol Delivery.”

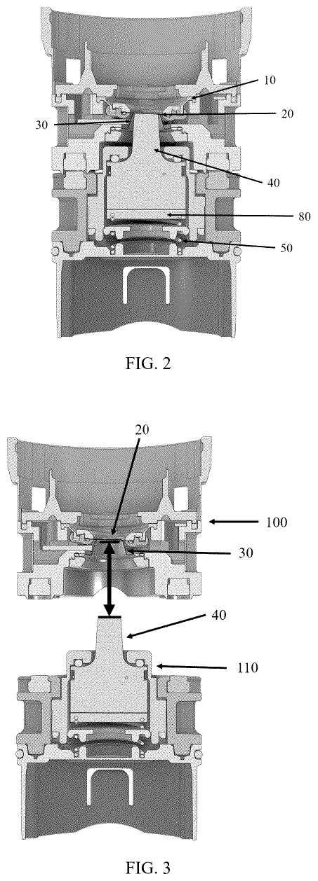

With an aerosol device that has a vibrating member (40), also known as a horn, in contact with an ejector plate (20), the force with which the vibrating member is pushing against the ejector plate is pivotal. This is the case with the “push mode” device. An example of a push mode device is shown in FIG. 2. The vibrating member has a piezoelectric transducer (80) (piezo) attached to it. The piezo transducer converts alternating electric field energy to mechanical vibrational energy. These vibrations are transferred to the vibrating member, then they are transferred to the ejector plate. The vibrations of the horn and ejector plate work in conjunction to generate the aerosol. The vibrating member is separated from the ejector plate by a membrane (30).

FIG. 3 illustrates how the ejector assembly (100) moves onto the vibrating member assembly (110) in a push mode design. The tip of the vibrating member (40) contacts the membrane (30)/ejector plate (20) interface.

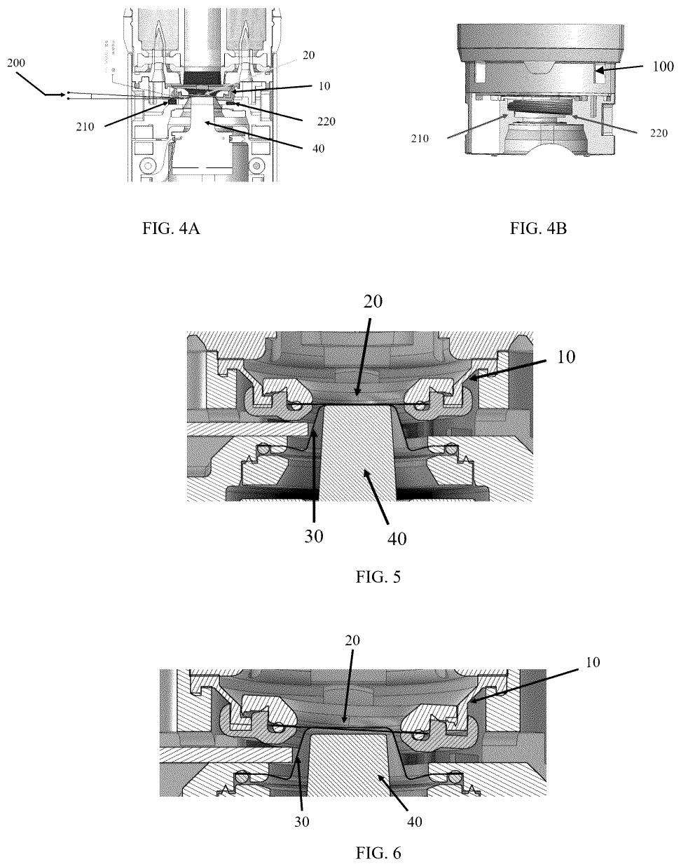

In a preferred example of the “push mode” device, there is a three-degree angle (200) to the ejector plate (20), which is shown in FIG. 4. The three-degree angle is created by a higher stop (210) and a lower stop (220), which force the suspension gasket (10) holding the ejector plate into a three-degree angle. This makes the tip of the vibrating member (40) contact the ejector plate at one point. This allows liquid to move freely between the vibrating member and the ejector plate. It also means that the vibrating member does not impede the vibrations of the ejector plate.

There is a range of contact forces at which aerosol can be generated. Anything above and below this force range causes ejection to not occur or be limited. Starting with no contact force between the ejector plate (20) and vibrating member (40), the vibrations cannot transfer to the ejector plate and, therefore, there will be no ejection. An example of little to no contact force between the vibrating member and ejector plate is shown in FIG. 6. Starting initial contact force allows some vibrations to transfer. Some ejection will happen, but it may not be an optimal amount. Increasing the contact force will allow vibrations to transfer more efficiently. This results in more ejection. Increasing the contact force past the highest ejection point results in interference. The vibrating member pushes too much onto the ejector plate, causing the vibrations of the ejector plate to be hindered by the vibrating member and membrane (30). Increasing the force even further can cause ejection to cease because the vibrating member is fully in contact with the ejector plate, inhibiting all vibrations. FIG. 5 shows an example of too much contact force between the vibrating member and ejector plate. In FIG. 5, the vibrating member tip is completely flat against the membrane, inhibiting vibrations. Obtaining the optimum force is important in achieving maximum ejection.

Additionally, contact force can influence the lifetime of the ejector plate and the membrane. When the force causes the membrane to be taut, vibrations transfer easily to the membrane. This causes the membrane to vibrate, heat up, and break. The contact force on the ejector plate can cause it to break sooner.

The goal is to provide just enough force to achieve enough ejected volume, but not so much force that it impedes the life span of the ejector plate (20) or membrane (30).

There are mechanisms put in place to achieve optimum contact force.

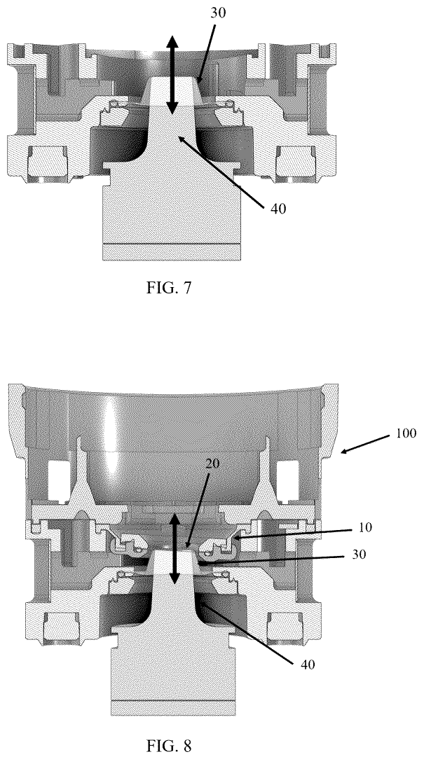

First, eliminating any unwanted tolerance issues in manufacturing the parts to ensure tight force tolerance when all the parts are put t together by good manufacturing processes and quality control. This includes, but is not limited to, machining the vibrating member (40) with a Swiss Screw Machine and implementing quality control measures. One of the quality control steps includes measuring the tensile force applied to the membrane (30) before the ejector assembly (100) is fully assembled. FIG. 7 illustrates the measurement of tensile force against the membrane as a quality check in a push mode device. In the measurement shown in FIG. 7, the ejector plate (20) is not yet assembled into the ejector assembly. In the measurement shown in FIG. 7, a vibrating member tip is forced onto the membrane at a known location and the force is measured. The next quality control step is measuring the tensile force applied to the membrane and ejector plate after the ejector assembly is fully assembled with the ejector plate. FIG. 8 illustrates the measurement of tensile force against the membrane and ejector plate as a quality check in a push mode device. A vibrating member tip is forced onto the ejector assembly at a known location and the force is measured.

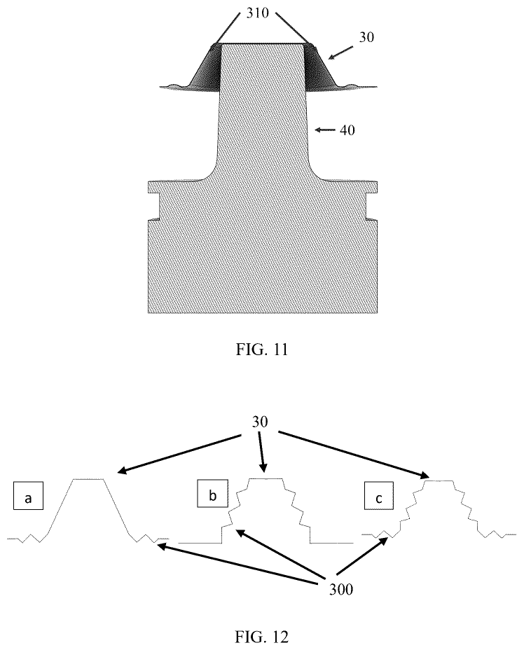

In another example to facilitate optimum contact force, the design of the membrane (30) helps eliminate unwanted additional forces applied to the vibrating member. The membrane should act as a barrier and any additional force applied to the vibrating member (40) is unwanted. This additional force could shorten the lifespan of the membrane. In this case, the membrane would be pulled taut, and vibrations would be transferred to the membrane increasing the energy transfer to the membrane. FIG. 11 illustrates the location on the membrane that gets too tight when there is too much contact force between the vibrating member and the membrane. This tight point (310) will experience excess vibrations and increased friction. The increase in friction will cause heat and will damage the membrane. Limiting the contact force is necessary for the longevity of the membrane.

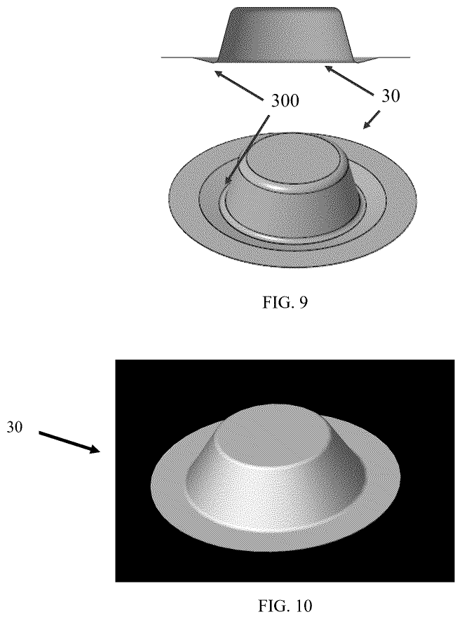

In a preferred example, which is shown in FIG. 9, the design of the membrane (30) includes additional material (300) such that the tip of the vibrating member (40) can be extended without the membrane pushing back on the vibrating member. The extra material is similar to a step in an accordion. In a non-limiting example the design can be accomplished by thermoforming the material. FIG. 10 shows an example of a membrane without the additional material/accordion step design.

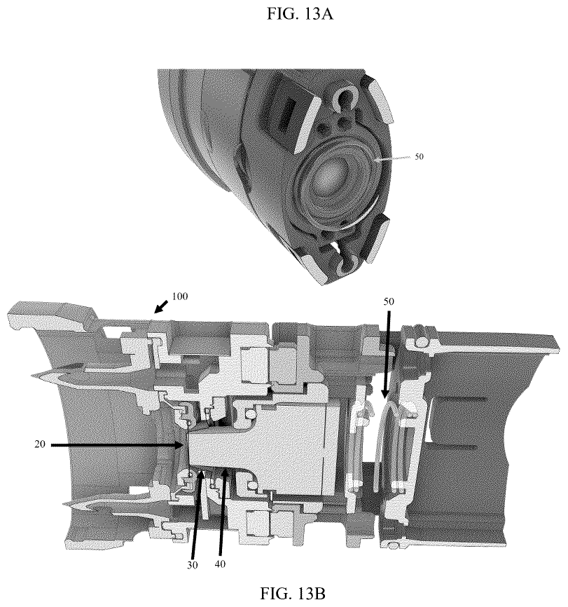

In another example, the membrane (30) has several accordion steps along its base (see FIG. 12a).

In another example, the membrane (30) has several accordion steps along its wall (see FIG. 12b).

In another example, the membrane (30) has several accordion steps along both the wall and its base (see FIG. 12c).

In a preferred example, the membrane (30) is made out of PEEK.

In other examples, the membrane (30) is made out of PEN, PPSU, PSU, Kapton, PTFE, ETFE, ABS, UHMW film, FEP, PFA, ECTFE, HDPE, TPE, polyester (PET), nylon, PEI, PPS, PVDF, UHMW, acetal (polyoxymethylene) POM, Polymethylpentene, or any other material that can be made around 30 microns thick.

In the preferred example, the membrane (30) is 30 microns thick.

In other examples the membrane (30) can be between 5 microns and 0.5 mm thick.

See Table 1 for data that helped the decision in which material and thickness to use for the membrane (30). Tests were done to measure the mass ejection of the device with two membrane materials (PEEK and PEN), three different thicknesses of material for each (16 μm, 25 μm, and 30 μm (PEEK) or 38 μm (PEN)), and two different designs. The designs either had the extra material for the step (FIG. 9), or did not (FIG. 10). There was a clear difference between the PEEK and PEN material. PEEK performed much better than PEN. There was little difference between the designs or the thicknesses. In theory, the design in FIG. 9 and thicker material should last longer than the other design and thinner material.

TABLE 1
The table below shows data that was used to help decide
which material and thickness to use for the membrane. The
PEEK material performed better than the PEN material.

| | Thickness of | | Average mass | STD | CV |
|---|---|---|---|---|---|
| Material | material (μm) | Design | ejection (mg) | (mg) | (%) |
| PEEK | 16 | Without step | 7.510 | 0.539 | 0.072 |
| PEEK | 25 | Without step | 7.980 | 0.689 | 0.086 |
| PEEK | 30 | Without step | 7.737 | 0.523 | 0.068 |
| PEEK | 16 | With step | 7.860 | 0.391 | 0.052 |
| PEEK | 25 | With step | 7.894 | 0.463 | 0.057 |
| PEEK | 30 | With step | 7.688 | 0.421 | 0.058 |
| PEN | 16 | Without step | 5.527 | 0.470 | 0.086 |
| PEN | 25 | Without step | 5.738 | 0.420 | 0.072 |
| EN | 38 | Without step | 4.501 | 0.278 | 0.062 |
| PEN | 16 | With step | 7.473 | 0.382 | 0.051 |
| PEN | 25 | With step | 6.917 | 0.322 | 0.047 |
| PEN | 38 | With step | 6.651 | 0.350 | 0.052 |

In another example, a desiccant is added to the ejector assembly (100) near the membrane (30). If any liquid or moisture appears on this side of the membrane, from permeation or condensation, the desiccant will absorb the liquid or moisture. Since this is on the ejector assembly, the desiccant will be replaced each time the ejector assembly is replaced. The desiccant can be many different shapes, one of which could be a desiccant ring (500) to encompass the ejector assembly on the outside of the membrane. This example is shown in FIG. 18.

In another example, there is an airtight seal between the underside of the ejector assembly (100) and the handpiece. This will limit the amount of water vapor near the membrane. This can be in combination with a desiccant.

In another example to facilitate optimum contact force is through a spring (50) attached to the bottom of the vibrating member assembly (110) to help widen the dimensional tolerance of the parts. The spring is designed to move slightly and provide a similar force. This means that if the dimensions of the parts being assembled are not the same, the spring will ensure the same force is applied between the vibrating member (40) and the membrane (30)/ejector plate (20) interface. FIG. 13 illustrates a spring beneath the vibrating member.

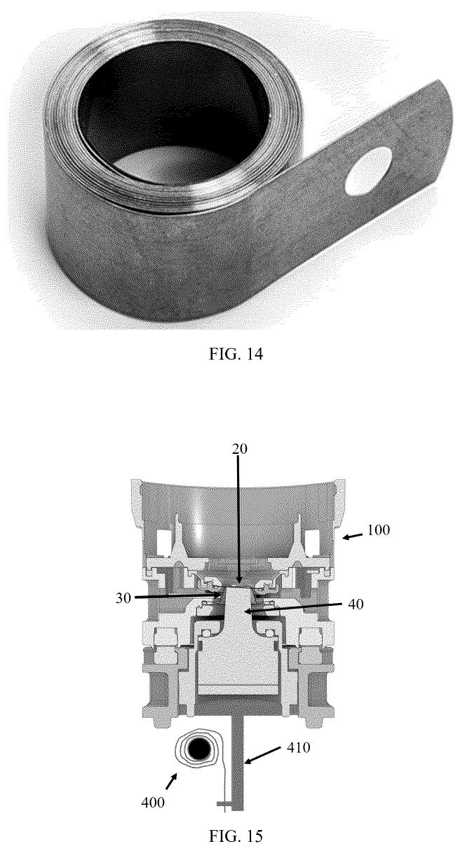

In one example, the spring is a “constant force spring.” This is a special type of spring that does not follow Hooke's Law. See FIG. 14 for a picture of a constant force spring (400) sold by Lee Spring. Once the constant force spring is set, the force with which the spring pushes back is the same no matter the distance it has moved.

FIG. 15 is an illustration of one way that the constant force spring (400) can be incorporated into the push mode design. An assembly (410) attached to the housing of the vibrating member is attached to the end of the constant force spring. The center of the constant force spring is attached to the handpiece. The constant force spring wants to coil itself back up. This forces the vibrating member (40) into the ejector assembly (100). The force which the spring provides does not change based upon the distance it is displaced.

In another example, two constant force springs (400) are used to evenly force the vibrating member (40) into the ejector plate (20).

In another example, more than two constant force springs (400) are used to evenly force the vibrating member (40) into the ejector plate (20).

In another example, the spring (50) is a compression spring. FIG. 13 illustrates an example of a push mode device with a compression spring. This spring does follow Hooke's Law. The spring would ideally have a very small spring constant. Therefore, the spring would supply a similar force with small changes in dimensions.

In a preferred example, the contact force is 0.85N. The contact force should be at least 0.6N. The contact force should be less than 1.2N. The optimal range is from 0.7N to 1.0N. The optimal range could change based on the material choices made. This includes the material for the ejector plate (20), suspension gasket (10) that holds the ejector plate, the membrane (30), the vibrating member (40), and housing plastic.

In another example, the optimal contact force range is between 0.5 N to 1.5 N.

In another example, the optimal contact force range is between 0.1 N to 0.5 N.

In another example, the optimal contact force range is between 0.1 N to 2.0 N.

In another example, the optimal contact force range is between 0.01 N and 0.5 N.

In another example, the optimal contact force range is between 1.0 N to 2.0 N.

In another example, the optimal contact force range is between 2.0 N to 5.0 N.

In another example, the optimal contact force range is between 5.0 N to 10.0 N.

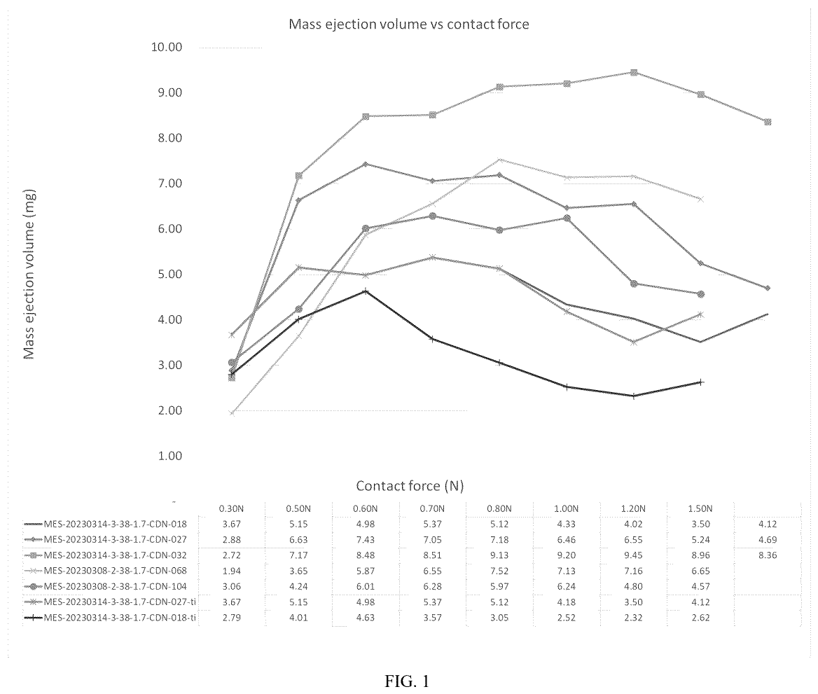

FIG. 1 shows data from a test to determine the optimum contact force for one example of a push mode design. Seven ejector sets were tested with varying contact force. The contact force ranged from 0.3 N to 1.5 N. The ejection volume started off low for every ejector at 0.3 N. The ejection volume increased with increasing contact force to a point. The point at which the ejected volume stopped increasing was between 0.6 N and 1.2 N. In most cases, any increase in ejection after 0.6 was minimal. The force 0.85 N was selected as the optimal force. A tolerance range of 0.15 N was selected to keep the contact force in the range of 0.7 N and 1.0 N. This ensures the contact for is near optimal performance without having too high of a force. A larger amount of contact force will result in lower ejected volume and lower membrane lifetime.

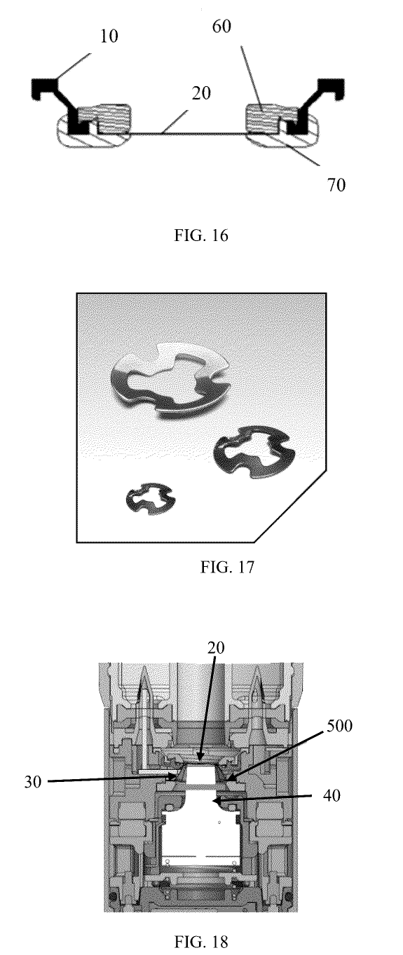

Another example to facilitate optimum contact force is with a silicone suspension gasket (10) that holds the ejector plate (20) in place. FIG. 16 illustrates an example of a silicone gasket and ejector plate. This gasket allows the ejector plate to get to a three-degree angle (200). It also keeps the ejector plate in contact with the vibrating member (40) but does not force the ejector plate into the vibrating member. Different durometer silicones can be used for the gasket. In a preferred example, 45 durometer is used. In other examples, 10, 20, 35, 40, 50, 60, or 70 durometer can be used.

In another example, a 0-degree angle is used for the ejector plate (20).

In another example, a 1-degree angle is used for the ejector plate (20).

In another example, a 2-degree angle is used for the ejector plate (20).

In another example, a 4-degree angle is used for the ejector plate (20).

In another example, a 5-degree angle is used for the ejector plate (20).

In another example, a 6-degree angle is used for the ejector plate (20).

In another example, a 7-degree angle is used for the ejector plate (20).

In another example, a 8-degree angle is used for the ejector plate (20).

In another example, a 9 to 12-degree angle is used for the ejector plate (20).

In another example, a 12 to 15-degree angle is used for the ejector plate (20).

In another example, a 15 to 20-degree angle is used for the ejector plate (20).

In another example, a 20 to 30-degree angle is used for the ejector plate (20).

In another example, a 30 to 45-degree angle is used for the ejector plate (20).

In another example, other materials are used for the suspension gasket (10) such as TPU, butyl rubber, etc.

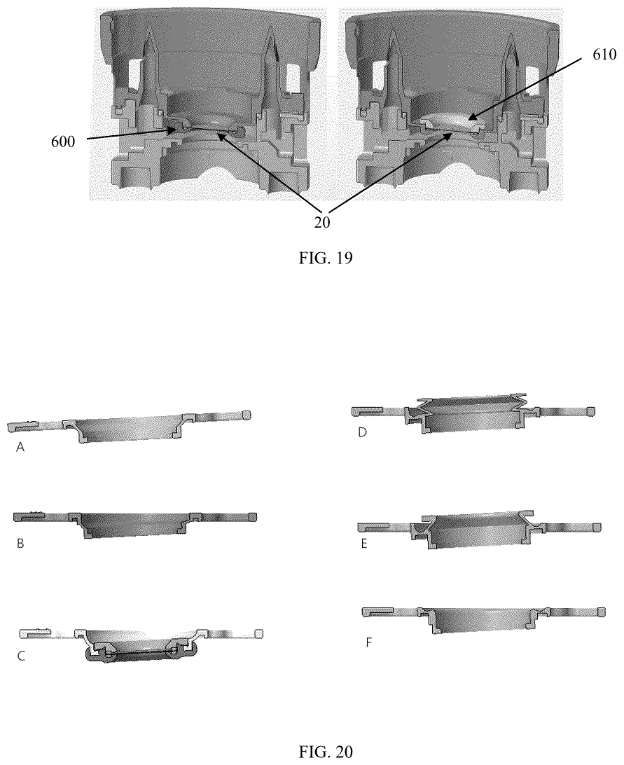

In another example the ejector plate (20) is fixed in place instead of having a suspension gasket (10). FIG. 19 illustrates an example of the fixed ejector plate. The ejector plate is fixed in place and does not move when the tip of the vibrating member (40) comes in contact with it. FIG. 19 illustrates two designs, one where a fixed bottom ring (600) holds the ejector plate in place and one where a fixed top ring (610) holds the ejector plate in place. A fixed ejector plate could eliminate some unwanted movement from the suspension gasket. A fixed ejector plate could reduce the number of parts and or steps to assembling the ejector assembly (100). This could increase repeatability.

In another example, the ejector plate (20) is horizontal and the vibrating member (40) has the three-degree angle cut into its tip. Refer to U.S. Pat. No. 11,793,945 for more detail on this concept and further examples.

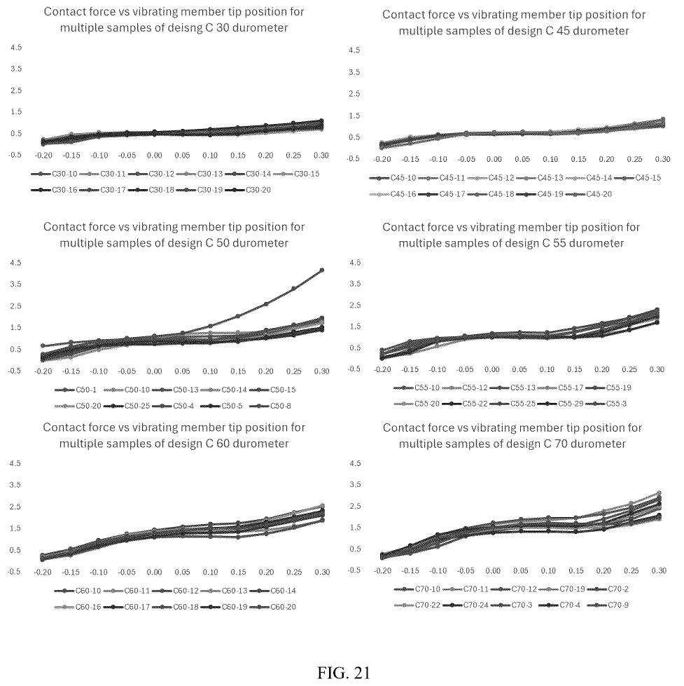

In another example, there is no spring (50) behind the vibrating member (40) and the suspension gasket (10) doubles as a spring, in addition to acting as a gasket. The suspension gasket can have different designs to change its compression and spring constant. The durometer of the silicone can change the compression and spring constant as well. FIG. 20 illustrates examples of different silicone suspension gaskets that could act as springs. The suspension gasket can be made of any squishy material. The durometer can be between 10 to 80. FIG. 21 shows data collected with design C from FIG. 20 using different durometer silicones. The data illustrates a reason why 45 durometer is a preferred example. A softer material has a more consistent contact force. However, softer material will also dampen any vibrations. Therefore, the durometer of the material must be hard enough to not dampen the vibrations, but soft enough to keep the contact force consistent.

In another example, the optimum contact force is between 0.1 N and 2.0 N.

In another example, a flat spring (thin round piece of metal) is connected to the suspension gasket (10) and is used as the mechanism to ensure proper force between the vibrating member (40) and the ejector plate (20). See FIG. 17 for an example of this type of flat spring. The design can be different. The type of metal can be any metal or metal alloy.

In another example a flat spring (FIG. 17) encompasses the vibrating member and is used as the mechanism to ensure proper force between the vibrating member (40) and the ejector plate (20). The design of the flat spring in FIG. 17 is for demonstration purposes and can be different. The flat spring can be made from any metal, metal alloy, or plastic.

Element numbers are provided in Table 2 for convenient reference with respect to the descriptions and figures provided herein.

TABLE 2
Element numbers and descriptions

| Number | Description |
|---|---|
| 10 | Suspension gasket |
| 20 | Ejector plate |
| 30 | Membrane |
| 40 | Vibrating member |
| 50 | Spring |
| 60 | Upper carrier |
| 70 | Lower carrier |
| 80 | Piezoelectric transducer |
| 100 | Ejector assembly |
| 110 | Vibrating member assembly |
| 200 | Three-degree angle |
| 210 | Higher stop |
| 220 | Lower stop |
| 300 | Extra material/accordion step |
| 310 | Tight point |
| 400 | Constant force spring |
| 410 | Assembly to attached vibrating member to constant force spring |
| 500 | Desiccant ring |
| 600 | Fixed bottom ring |
| 610 | Fixed top ring |

While described with reference to specific examples herein, the invention is intended to extend in scope to the full extent of the claims.

What is claimed is:

1. An aerosol delivery device comprising:
a piezoelectric transducer indirectly coupled to an ejector plate; and
a suspension gasket coupled to the ejector plate that is configured to apply spring force between the ejector plate and the transducer.

2. An aerosol delivery device comprising:
a piezoelectric transducer indirectly coupled to an ejector plate; and
a compression spring coupled to the piezoelectric transducer and configured to provide a spring force between the ejector plate and the transducer.

3. The device of claim 2, wherein the ejector plate is held by a suspension gasket configured to provide additional spring force between the ejector plate and the transducer.

4. The device of claim 2, wherein the ejector plate is rigidly held in place and wherein only the compression spring is providing spring force.

5. An aerosol delivery device comprising:
a piezoelectric transducer indirectly coupled to an ejector plate; and
a flat spring coupled to the ejector plate that is configured to apply contact force between the ejector plate and the transducer.

6. An aerosol delivery device comprising:
a piezoelectric transducer indirectly coupled to an ejector plate; and
one or more constant force springs coupled to the piezoelectric transducer and configured to provide a contact force between the ejector plate and the transducer.

7. The device of claim 6 wherein the ejector plate is held by a suspension gasket configured to provide additional spring force between the ejector plate and the transducer.

8. The device of claim 6 wherein the ejector plate is rigidly held in place wherein only the constant force spring or constant force springs provide spring force.

9. An aerosol delivery device comprising a piezoelectric transducer indirectly coupled to an ejector plate via a membrane between the transducer and the plate, wherein the membrane includes folds configured to control contact force on the membrane.

10. An aerosol delivery device comprising:
a piezoelectric transducer indirectly coupled to an ejector plate via a membrane between the transducer and the plate; wherein spring force is being applied between the ejector plate and the transducer;
a vibrating member between the membrane and the plate; and
a desiccant placed near the vibrating member that is configured to absorb unwanted moisture.

11. The delivery device of claim 10, wherein the desiccant is housed in a location sealed from the environment.
